(19) United States
(12) Patent Application Publication
(10) Pub. No.: US 2022/0168150 A1
(43) Pub. Date: Jun. 2, 2022

(54) SYSTEM AND METHODS OF USE FOR PRE-CUT KINESIOLOGY TAPE EMBEDDED WITH MICROENCAPSULATED ESSENTIAL OILS

(71) Applicant: Dermafuse, LLC, Nephi, UT (US)

(72) Inventors: John Wesley WILLMORE, Nephi, UT (US); James Franklin ROSENBECK, Nephi, UT (US); Steven Richard ROSENBECK, Nephi, UT (US)

(73) Assignee: Dermafuse, LLC, Nephi, UT (US)

(21) Appl. No.: 17/436,543

(22) PCT Filed: Mar. 5, 2020
(86) PCT No.: PCT/US2020/021156

§ 371 Date: Sep. 3, 2021

Related U.S. Application Data
(60) Provisional application No. 62/814,148, filed on Mar. 5, 2019.

(51) Int. Cl. A61F 13/00; A61F 13/02

(52) U.S. Cl. CPC A61F 13/00063; A61F 13/0283; A61F 13/025

(57) ABSTRACT

A pre-cut piece of kinesiology tape is provided, the pre-cut piece of kinesiology tape having a shape that facilitates a predetermined purpose (such as pain relief, inflammation reduction, muscle inhibition, etc.). The pre-cut piece of kinesiology tape comprises a flexible fabric layer and an adhesive layer. The fabric layer is embedded with microencapsulated essential oils.

### TECHNICAL FIELD

The present invention relates generally to an application of kinesiology tape. More specifically, the present invention relates to systems and methods of kinesiology tape which may be embedded with microencapsulated essential oils.

### BACKGROUND

Kinesiology tape may be generally used to restrict an individual's body movement or provide a force on the skin to assist in muscle inhibition, muscle facilitation, and/or muscle correction. Kinesiology tape may be used to treat injuries or to prevent injuries in muscles and/or joints. Proper placement of the kinesiology tape, including proper stretching of the tape prior to placement, is important for the kinesiology tape to provide the intended benefits. Many times, kinesiology tape is dispensed in a roll and placed by a professional such as a physical therapist for proper placement. In other instances, kinesiology tape is placed by non-professionals guided by instructions or assisted by pre-cut tape configurations.

Essential oils may also be used for treating and/or preventing injuries. Thus, it may be desirable to combine a kinesiology tape with essential oils for treating and/or preventing injuries. It also may be desirable to provide the tape in a manner which simplifies proper placement such that a user may place the tape themselves rather than needing a professional to place the tape. The tape may have other uses, including pain relief, headache relief, etc.

### SUMMARY OF INVENTION

According to one aspect of the present disclosure, a kinesiology tape is provided. The tape may be pre-cut in a particular shape to achieve a desired result. In other instances, the tape may be provided on a continuous roll. In others, the tape may be provided in precut strips. Such precut strips may be provided individually, or they may be provided in roll form. In some instances, the strips may be attached to a backing or carrier material and either spaced apart or separated by complete or discontinuous cuts or perforations.

The disclosed kinesiology tape may comprise a fabric layer and an adhesive layer. The fabric layer may comprise a weave of fibers that is somewhat flexible. For example, some or all of the fibers may comprise elastic fibers covered in a covering such as cotton or another material. The flexibility of the fabric layer may allow the tape to be applied in a manner that restricts muscle movement, lifts muscles, etc. The adhesive layer may comprise one or more strips of adhesive. In some instances, the strips of adhesive may cover all, most or at least substantially all of a skin-facing face of the fabric layer. In others, the strips of adhesive may be narrower than the fabric layer. In such embodiments of the kinesiology tape, when in use, at least a portion of the fabric layer may be in direct contact with a user's skin. In other configurations the fabric layer need not be in direct contact with a user's skin.

The fabric layer of the kinesiology tape of the present disclosure is embedded with microencapsulated essential oils. In some instances, the oils may be selected for their ability to complement the desired results of the particular application of the kinesiology tape.

According to one configuration, a method is provided to treat muscle pain, wherein a kinesiology tape, such as, in some instances, a precut kinesiology tape, is selected for its ability to treat muscle pain, and wherein the fabric layer of the kinesiology tape is treated with microencapsulated essential oils selected for their ability to treat muscle pain, and the kinesiology tape is applied to a user in need thereof.

According to another configuration, a method is provided to facilitate muscle inhibition, wherein a kinesiology tape, such as, in some instances, a precut kinesiology tape, is selected for its ability to facilitate muscle inhibition, and wherein the fabric layer of the kinesiology tape is treated with microencapsulated essential oils selected for their ability to reduce tension, and the kinesiology tape is applied to a user in need thereof.

According to one configuration, a method is provided to aid in muscle recovery, wherein a kinesiology tape, such as, in some instances, a precut kinesiology tape, is selected for its ability to treat muscle pain, and wherein the fabric layer of the kinesiology tape is treated with microencapsulated essential oils selected for their ability to aid in muscle recovery, and the kinesiology tape is applied to a user in need thereof.

### BRIEF DESCRIPTION OF DRAWINGS

The following drawings illustrate what are currently considered to be specific representative configurations for carrying out the invention and are not limiting as to embodiments which may be made in accordance with the present invention. The components in the drawings are not necessarily to scale relative to each other. Like reference numerals designate corresponding parts throughout the several views.

The drawings are illustrative and not limiting of the scope of the invention which is defined by the appended claims. The various elements of the invention accomplish various aspects and objects of the invention. Not every element of the invention can be clearly displayed in a single drawing, and as such not every drawing shows each element of the invention.

FIG. 1 is an end view of one configuration of kinesiology tape.

FIG. 2 is a bottom plan view of one configuration of kinesiology tape showing an exemplary adhesive layer.

FIG. 3 is a top plan view of one configuration of kinesiology tape having a pre-cut U-shape.

FIG. 4 is a top plan view of one configuration of kinesiology tape having a pre-cut X-shape.

FIG. 5 is a top plan view of one configuration of kinesiology tape having a pre-cut V-shape.

FIG. 6 is a top plan view of one configuration of kinesiology tape having a pre-cut L-shape.

FIG. 7 is a top plan view of one configuration of kinesiology tape having a pre-cut oval shape with one or more cut-outs.

FIG. 8 is a top plan view of one configuration of kinesiology tape having a pre-cut Y-shape.

FIG. 9 is a schematic view showing a portion of the fabric layer with the microcapsules embedded in the fabric layer.

FIG. 10 is a detailed schematic view showing a portion of the fabric layer with the microcapsules embedded in the fabric layer in the background, and an enlarged, opened microcapsule in the foreground.

FIG. 11 is a top plan view of one configuration of the present disclosure that may be used to reduce nasal congestion.

FIG. 12 is a top plan view of one configuration of the present disclosure that may be used to reduce pain and tension from a headache.

FIG. 13 is a top plan view of one configuration of the present disclosure that may be used to treat the neck/upper back.

FIG. 14 is a top plan view of one configuration of the present disclosure that may be used to treat the low back.

FIG. 15 is a top plan view of one configuration of the present disclosure that may be used to treat shin splints.

FIG. 16 is a top plan view of one configuration of the present disclosure that may be used to treat muscle pain.

### DETAILED DESCRIPTION

The following provides a detailed description of particular embodiments of the present invention. Reference will now be made to the drawings in which the various elements of the illustrated configurations will be given numerical designations and in which the invention will be discussed to enable one skilled in the art to make and use the invention. It is to be understood that the following description is only exemplary of the principles of the present invention, and should not be viewed as narrowing the scope of the claims which follow, which claims define the full scope of the invention.

It will be appreciated that various aspects discussed in one drawing may be present and/or used in conjunction with the embodiment shown in another drawing, and each element shown in multiple drawings may be discussed only once. For example, in some cases, a detailed description of well-known items or repeated description of substantially the same configurations may be omitted to avoid repetition.

It should be noted that the description merely illustrates the principles of the present subject matter. It will thus be appreciated that those skilled in the art will be able to devise various arrangements that, although not explicitly described herein, embody the principles of the present subject matter and are included within its spirit and scope. Furthermore, all examples recited herein are principally intended expressly to be only for pedagogical purposes to aid the reader in understanding the principles of the invention and the concepts contributed by the inventor(s) to furthering the art, and are to be construed as being without limitation to such specifically recited examples and conditions. Moreover, all statements herein reciting principles, aspects, and embodiments of the invention, as well as specific examples thereof, are intended to encompass equivalents thereof.

Reference in the specification to “one configuration” “one embodiment,” “a configuration” or “an embodiment” means that a particular feature, structure, or characteristic described in connection with the configuration is included in at least one configuration, but is not a requirement that such feature, structure or characteristic be present in any particular configuration unless expressly set forth in the claims as being present. The appearances of the phrase “in one configuration” in various places may not necessarily limit the inclusion of a particular element of the invention to a single configuration, rather the element may be included in other or all configurations discussed herein.

Furthermore, the described features, structures, or characteristics of configurations of the invention may be combined in any suitable manner in one or more configurations. In the following description, numerous specific details are provided, such as examples of products or manufacturing techniques that may be used, to provide a thorough understanding of configurations of the invention. One skilled in the relevant art will recognize, however, that configurations of the invention may be practiced without one or more of the specific details, or with other methods, components, materials, and so forth. In other instances, well-known structures, materials, or operations are not shown or described in detail to avoid obscuring aspects of the invention.

Before the present invention is disclosed and described in detail, it should be understood that the present disclosure is not limited to any particular structures, process steps, or materials discussed or disclosed herein, but is extended to include equivalents thereof as would be recognized by those of ordinary skill in the relevant art. More specifically, the invention is defined by the terms set forth in the claims. It should also be understood that terminology contained herein is used for the purpose of describing particular aspects of the invention only and is not intended to limit the invention to the aspects or configurations shown unless expressly indicated as such. Likewise, the discussion of any particular aspect of the invention is not to be understood as a requirement that such aspect is required to be present apart from an express inclusion of the aspect in the claims.

It should also be noted that, as used in this specification and the appended claims, singular forms such as “a,” “an,” and “the” may include the plural unless the context clearly dictates otherwise. Thus, for example, reference to “an adhesive” may include one or more of such adhesives, and reference to “the essential oil” may include reference to one or more of such essential oils.

As used herein, the term “substantially” refers to the complete or nearly complete extent or degree of an action, characteristic, property, state, structure, item, or result to function as indicated. For example, an essential oil that is “substantially” encapsulated would mean that the object is either completely encapsulated or nearly completely encapsulated. The exact allowable degree of deviation from absolute completeness may in some cases depend on the specific context, such that encapsulated nearly all of an essential oil would be substantially encapsulated, even if part of the structure encapsulating the essential oil had opening formed along a portion thereof. The use of “substantially” is equally applicable when used in a negative connotation to refer to the complete or near complete lack of an action, characteristic, property, state, structure, item, or result. For example, structure which is “substantially free of” adhesive would either completely lack adhesive or so nearly completely lack adhesive that the effect would be effectively the same as if it lacked adhesive.

As used herein the term “generally” refers to something that is more of the designated adjective than not, or the converse if used in the negative. For example, something maybe said to be generally circular even though it has a somewhat oval shape or is polygonal rather than being completely smooth.

As used herein, the term “about” is used to provide flexibility to a numerical range endpoint by providing that a given value may be “a little above” or “a little below” the endpoint while still accomplishing the function associated with the range.

As used herein, a plurality of items, structural elements, compositional elements, and/or materials may be presented in a common list for convenience. However, these lists should be construed as though each member of the list is individually identified as a separate and unique member.

Concentrations, amounts, proportions and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

The present invention generally relates to a system and method of use for kinesiology tape, wherein the tape may be provided in rolls for cutting and/or customized applications, or may be pre-cut into formations or strips that allow for ease of application, and wherein the tape is embedded with one or more essential oils which may be microencapsulated to protect the one or more essential oils during storage, and to allow release during use. In other configurations the micro-encapsulated essential oils may be provided on a roll of kinesiology tape.

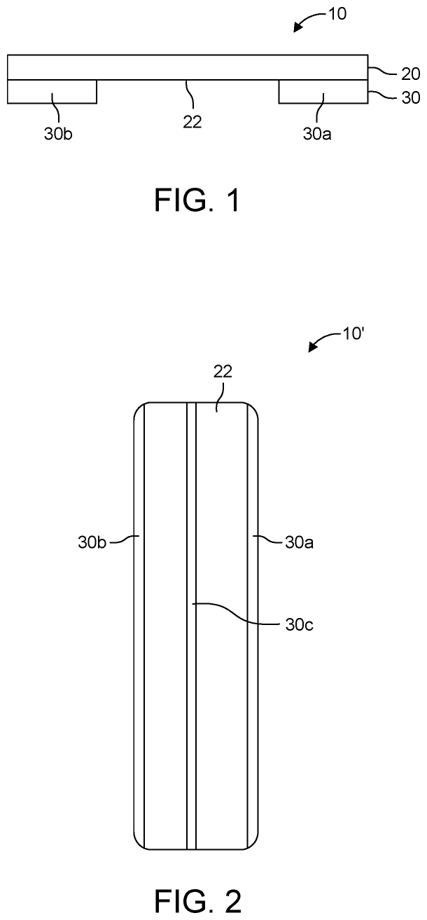

One particular embodiment of the present disclosure is shown and described in a kinesiology tape strip 10 of FIG. 1. The kinesiology tape 10 of FIG. 1 may include a fabric layer 20 and an adhesive layer 30. The fabric layer 20 may comprise a weave of fibers that is somewhat flexible. For example, some or all of the fibers may comprise elastic fibers covered in a covering such as cotton or another material. The flexibility of the fabric layer 20 may allow the tape to be applied in a manner that restricts muscle movement, lifts muscles, etc.

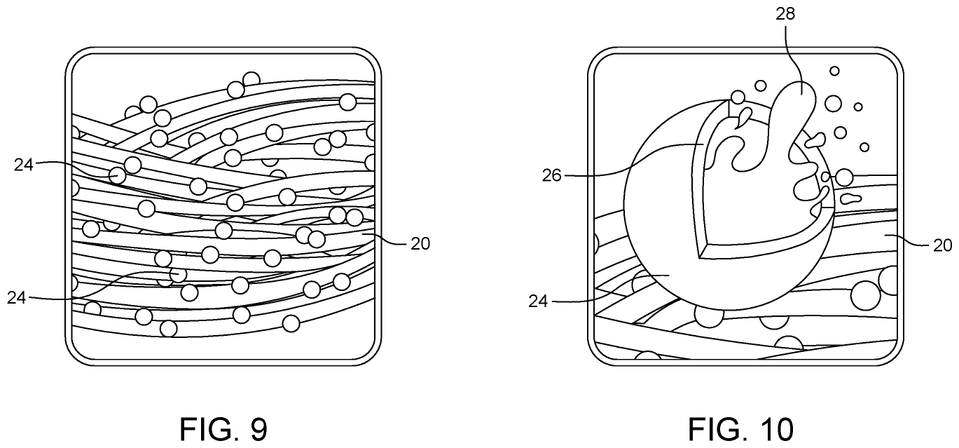

The fabric layer 20 may further comprise one or more micro-encapsulated essential oils (not visible in FIG. 1). Microencapsulation is the process that one material or a mixture of materials is coated with or entrapped within another material or system. The coated material is called active or core material, and the coating material is called shell, wall material, carrier or encapsulating agent. As used herein the term “microencapsulated” refer to microcapsules having a shell and core structure, specifically a solid shell surrounding a core comprising temporarily entrapped substances such as one or more essential oils. FIG. 9 shows a schematic view of a portion of the fabric layer 20 loaded or embedded with a plurality of microcapsules 24, each microcapsule including an essential oil or mixture or oils for release during use. FIG. 10 shows a schematic of a portion of the fabric layer 20 with the microcapsules 24 embedded in the fabric layer in the background, and an enlarged, opened microcapsule 24 in the foreground. The opened microcapsule allows the microcapsule shell or wall 26 to be visible, as well as the entrapped essential oil 28.

The essential oils may be micro-encapsulated according to any suitable method known in the art, including physical methods, physicochemical methods, and chemical methods. Some exemplary methods include simple or complex coacervation, spray drying, spray chilling, fluidized bed spray coating, emulsification, interfacial polymerization (such as polycondensation or polyaddition from dispersions), in situ polymerization and/or others known to one of ordinary skill in the art. For example, WO 2004/098767, incorporated herein in its entirety, discloses a process for the preparation of essential oil microcapsules by encapsulation of an essential oil through interfacial polymerization, whereby a polyurea and/or polyurethane film is formed around the liquid essential oil droplets. Similarly, U.S. Pat. No. 8,753,676, incorporated herein in its entirety, discloses a method for the preparation of microencapsulated essential oils. Microencapsulation of essential oils can protect and prevent the loss of volatile aromatic ingredients prior to use and may improve the controlled release and stability of the essential oils. Manufacturers including Encapsys are known to practice suitable methods for encapsulation of oils.

Such methods may be used to encapsulate the essential oils used in the preparation of the kinesiology tapes of the present invention. Some technologies for the encapsulation of oils are described in U.S. Pat. No.: 10,485,739, U.S. Pat. No.: 10,456,766, U.S. Pat. No.: 10,428,294, U.S. Pat. No.: 10,308,894, U.S. Pat. No.: 10,292,910, U.S. Pat. No.: 10,208,428, U.S. Pat. No.: 9,896,649, U.S. Pat. No.: 9,809,725, U.S. Pat. No.: 9,714,397, U.S. Pat. No.: 9,714,396, U.S. Pat. No.: 9,656,233, U.S. Pat. No.: 9,528,032, U.S. Pat. No.: 9,205,395, and U.S. Pat. No.: 9,192,908, each of which is incorporated by reference in its entirety.

According to some embodiments, the term “microcapsule” refers to microcapsules having an average size of 0.05-250 microns, 0.1-150 microns, 1-100 microns, 10-100 microns, 20-100 microns or any other range within 0.05-250 microns. Among other benefits, microencapsulation can protect a core substance from the effects of UV rays, moisture and oxygen; increase the storage life of a volatile compound; decrease the rate of evaporation or transfer of the active material from the core to the medium; improve the handling properties of sticky materials; and control release of core material.

In some configurations, the microcapsule comprises 10-20, 20-30, 30-40, 40-50, 50-60, 60-70, 70-80, 80-90, 90-95 or any other suitable range within the range of 10-95 percent per weight essential oil(s). In some configurations it may be preferred that the microcapsule comprises above 20 percent per weight essential oil(s). The remainder of the microcapsules may be comprised of the encapsulating shells and additives. In other configurations, no additives are used. Encapsulating shells may be formed of any suitable material depending on the desired type of release, such as gum arabic and maltodextrin, alginate, chitosan, gelatin, ethylcellulose, melamine formaldehyde, and/or polymethyl methacrylate.

The microencapsulated essential oil(s) may be embedded in the fabric layer, for example, by spraying one or both sides of the tape, soaking or otherwise contacting the fabric layer with a suspension, such as an aqueous suspension, of micro-encapsulated essential oil(s). The step of embedding the microencapsulated essential oil(s) in the fabric layer may be conducted before, or in some instances, after, the application of the adhesive layer to the fabric layer. Alternatively, or in addition to embedding the microencapsulated essential oil(s) in the fabric layer, the microencapsulated essential oil(s) may be mixed into the adhesive used to prepare the adhesive layer of the kinesiology tape. In some configurations, the concentration of the encapsulated essential oil within the fabric is in the range of 0.1 gr of encapsulated essential oil/m² to 20 gr of encapsulated essential oil/m² of fabric. In some configurations, the micro-particle wall material that forms the micro-capsule to encapsulate the essential oil(s) is formed of a material that breaks down when friction is applied to the fabric layer. This may allow effective release of the essential oil(s), for example, as a user applies the tape and again as the subject wearing the tape moves their body or applies friction or pressure to the tape.

In other configurations, the microparticle wall material may be formed of a material that breaks down when exposed to moisture. This may allow effective release of the essential oil(s) when sweat from a user's skin contacts the skin-facing side of the fabric layer, either directly or indirectly. In other configurations, the microparticle wall material may be formed of a material that breaks down when exposed to a higher temperature, such as when a user sweats and their skin temperature increases.

In some configurations, a method of micro-encapsulation may be used that allows the essential oils to be released over time. Microcapsules formed to have a controlled release profile in the fabric layer may ensure prolonged duration of the essential oil release as a method of treatment for the user. As some applications of kinesiology tape are intended to be worn for many hours, and in some cases, for multiple days, it may be advantageous for a user selecting a kinesiology tape to treat pain to experience a prolonged duration of pain relief due to release of essential oils throughout the entire period during which the tape is worn.

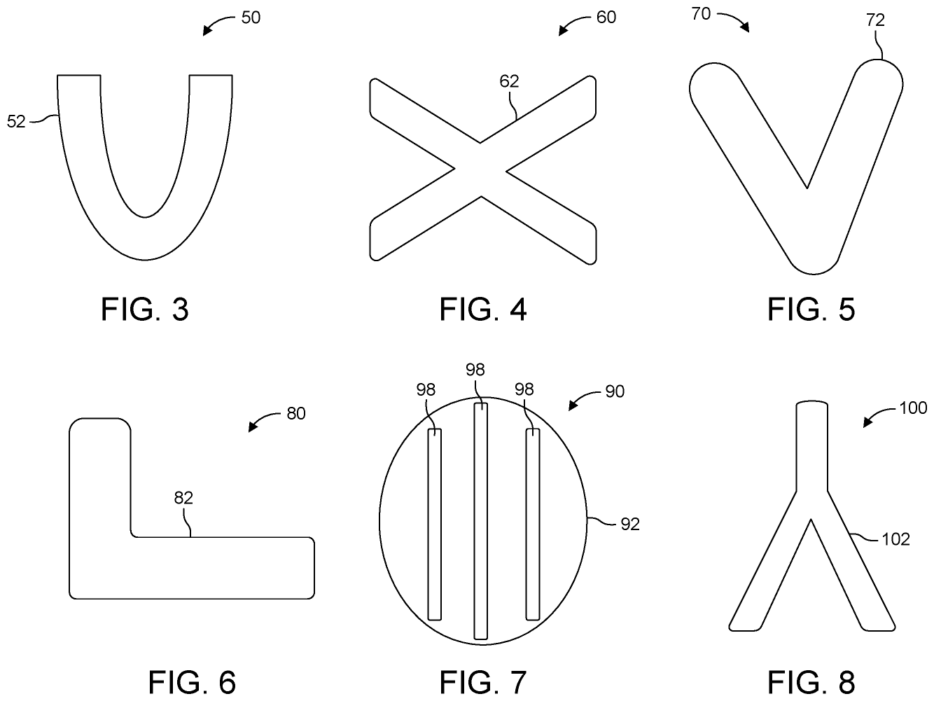

The kinesiology tape 10 may further include an adhesive layer 30, configured to adhere on one side to the skin-facing side 22 of the fabric layer 20, and on its other side to the skin of a user. In some configurations, the adhesive layer 30 comprises one or more strips of adhesive (30a, 30b of tape 10 in FIGS. 1 and 30a, 30b, 30c of tape 10′ in FIG. 2) connected to the fabric layer 20. The strips of adhesive may have a width substantially equal to, or in some instances, less than the width of the fabric layer, such that the adhesive layer does not cover the skin-facing side of the fabric layer entirely. In this manner the portions of the skin-facing side of the fabric layer that are not covered in adhesive may directly contact the skin of the user or may be proximal to the skin of the user. In some cases, direct contact may not be necessary for effective delivery of the microcapsules from the fabric layer to the skin of the user. It will further be appreciated that the one or more strips of adhesive of the adhesive layer may be formed in any configuration (such as the three straight strips 30a, 30b, 30c shown in FIG. 3, 4 or more strips, zig-zag shaped-strips, wavy strips, etc.).

According to another aspect, the kinesiology tape may be provided in rolls, either of continuous tape, or as pre-cut strips, or it may be provided in pre-cut shapes for pre-determined purposes/applications/placements. As discussed herein, the kinesiology tape provided in rolls of continuous tape, as pre-cut strips, or as pre-cut shapes may be further provided with micro-encapsulated essential oils to further aid the pre-determined purpose. FIGS. 3 through 8 illustrate exemplary shapes that may be used for pre-determined purposes. By providing the kinesiology tape in pre-cut forms, it may simplify application by users. Professionals such as physical or sports therapists may specialize in cutting strips of tape and applying them in configurations to achieve specific purposes, but it may be difficult for an average user without training to cut and apply tape for a specific purpose. The pre-cut shapes and strips may have rounded corners to encourage the kinesiology tape to stay on for a greater period of time and reduce fraying.

FIG. 3 shows a pre-cut U-shape 50 that may simplify placement of kinesiology tape around a knee. Without this pre-cut shape, typically three different strips of kinesiology tape would have to be placed to support the knee. This particular pre-cut U-shape 50 may help support the patella, reduce inflammation in the meniscus, and ease arthritis in the knee joint of a user. The fabric layer 52 of the pre-cut U-shape may be treated with one or more essential oils that reduce inflammation to further treat the knee.

According to another example, FIG. 4 depicts a pre-cut shape of kinesiology tape 60 generally in the shape of an “X.” This pre-cut shape may be selected for the predetermined purpose of targeted pain relief. The fabric layer 62 of this pre-cut shape 60 may be embedded with micro-encapsulated essential oils that are similarly chosen for the predetermined purpose of targeted pain relief. For example, the micro-encapsulated essential oils for this pre-cut kinesiology tape may be selected singly, or as a mixture from the group consisting of: rosehip, lavender, evening primrose, wintergreen, helichrysum, menthol, blue chamomile, flaxseed, Osmanthus, clary sage, ylang ylang, eucalyptus, rosemary, blue tansy, camphor, juniper berry, copaiba (such as copaifera reticulata, copaifera officinalis, copaifera langsdorffii and/or copaifera coriacea), chamomile, cayenne, sandalwood, clove, marjoram, yarrow, lemongrass, wintergreen, and peppermint.

FIG. 5 shows a pre-cut V-shape 70 that may simplify placement of kinesiology tape around the front or back of the knee. Without this pre-cut shape 70, typically two different strips of kinesiology tape would have to be placed to support the knee. This particular pre-cut U-shape 70 may help support the patella, reduce inflammation in the meniscus, and ease arthritis. The fabric layer 72 of the pre-cut V-shape may be treated with one or more essential oils that reduce inflammation to further treat the knee.

FIG. 6 shows a pre-cut L-shape 80 that may simplify placement of kinesiology tape on arms and elbows. Without this pre-cut shape, typically two to three different strips of kinesiology tape would have to be placed to support the elbow. This particular pre-cut L-shape 80 may help support the elbow and ease arthritis. The fabric layer 82 of the pre-cut L-shape 80 may be treated with one or more essential oils that reduce inflammation to further treat the elbow.

FIG. 7 depicts a pre-cut tape 90 having a circular or oval shape, and further having strips of cut-outs 98. This type of pre-cut tape 90 may be particularly useful in recovery to reduce swelling and inflammation. The fabric layer 92 of this shape 90 of pre-cut tape may be provided with micro-encapsulated essential oils selected for their ability to also reduce swelling and inflammation. For example, one or more essential oil(s) may be selected singly, or as a mixture from the group of: thyme, clove, rose, fennel, bergamot, peppermint, menthol, eucalyptus, lavender, German or Roman chamomile, spruce, ginger, patchouli, and helichrysum. It will be appreciated that in applications where more than one essential oil is selected, the essential oils may be first formed into a blend, and the blend may be encapsulated. Or, the individual essential oils may be encapsulated, and the microcapsules may be blended together.

Additionally, the pre-cut shape 90 of FIG. 7 is typically designed to be worn 2-3 days. The micro-encapsulated essential oil(s) embedded in the fabric layer 92 of the kinesiology tape 90 may similarly be designed to release essential oils over a period of 2-3 days to aid in reducing swelling and inflammation.

FIG. 8 shows a pre-cut Y-shape 100 that may simplify placement of kinesiology tape around the shoulder or the knee. Without this pre-cut shape 100, typically two to three different strips of kinesiology tape would have to be placed to support the knee. This particular pre-cut shape 100 may help support the knee/shoulder, reduce inflammation, and ease arthritis. The fabric layer 102 of the pre-cut Y-shape may be treated with one or more essential oils that reduce inflammation to further treat the knee/shoulder.

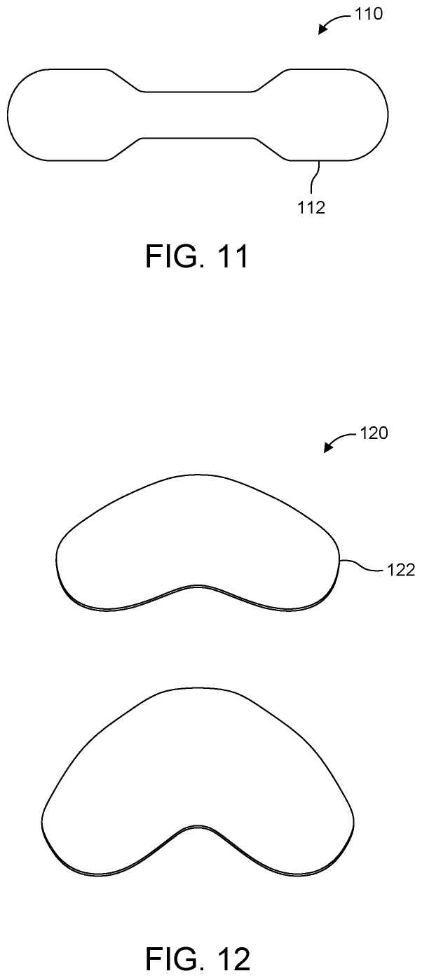

FIG. 11 is a plan view of one configuration of tape 110 of the present disclosure that may be used to reduce nasal congestion. Essential oils embedded on the fabric layer 112 of the pre-cut nasal congestion strip 110 (or on a roll of kinesiology tape that may be cut by the user for nasal congestion) may include one or more of the following, or other oils as desired: tea tree, eucalyptus, peppermint, menthol, and oregano.

FIG. 12 is a plan view of one configuration 120 of the present disclosure that may be used to reduce pain and tension from a headache. The kinesiology tape may be applied on the forehead, at the base of the neck, etc. Several different types of essential oils may be useful for the encapsulated oil for the fabric layer 122. For example, the micro-encapsulated essential oils for this pre-cut kinesiology tape may be selected from the group consisting of: rosehip, lavender, evening primrose, flaxseed, clary sage, eucalyptus, rosemary, juniper berry, chamomile, cayenne, sandalwood, clove, marjoram, yarrow, lemongrass, wintergreen, menthol, and peppermint.

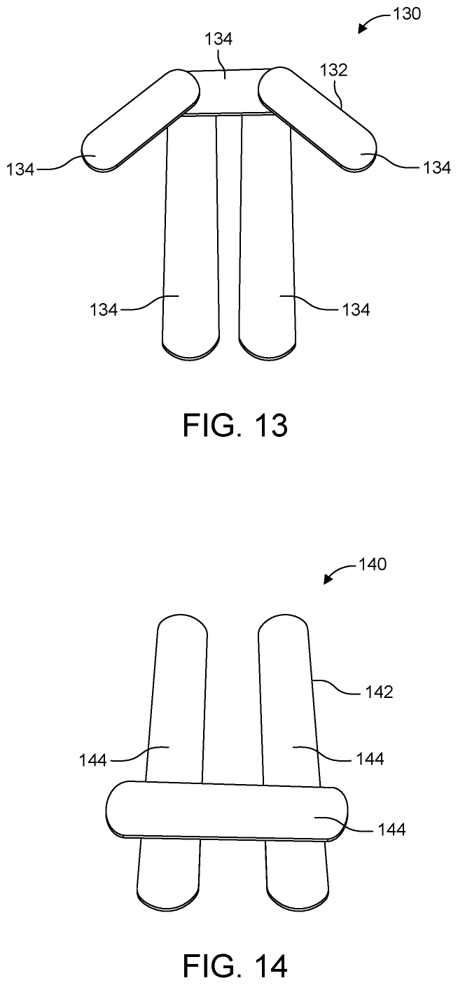

FIG. 13 is a plan view of one configuration 130 of the present disclosure that may be used to treat the neck/upper back. The kinesiology tape 130 may be provided as one single piece, or may be provided as a kit of one or more pre-cut strips (such as 5 strips 134 as shown in FIG. 13, though more or fewer strips may be provided of different shapes to approximate the shape illustrated in FIG. 13). Each strip 134 may be embedded with the same micro-encapsulated essential oil(s), or one or more strips may have different micro-encapsulated essential oil(s). The strips may also be provided with one or more colors to color-code the strips to simplify placement for the user. Several different types of essential oils may be useful for the encapsulated oil for the fabric layer 132. For example, the micro-encapsulated essential oils for this pre-cut kinesiology tape may be selected from singly, or as a mixture from the group consisting of: rosehip, lavender, evening primrose, flaxseed, clary sage, eucalyptus, rosemary, juniper berry, chamomile, cayenne, sandalwood, clove, marjoram, yarrow, lemongrass, wintergreen, and peppermint.

FIG. 14 is a plan view of one configuration 140 of the present disclosure that may be used to treat issues of the lower back. Similar to the configuration shown in FIG. 13, one or more strip(s) 144 of pre-cut tape may be provided (which may or may not be color-coded), or a single pre-cut shape may be provided. Depending on the desired effects, for example, for relaxation, pain relief, etc., various types of micro-encapsulated essential oils may be selected singly, or as a mixture for the fabric layer 142.

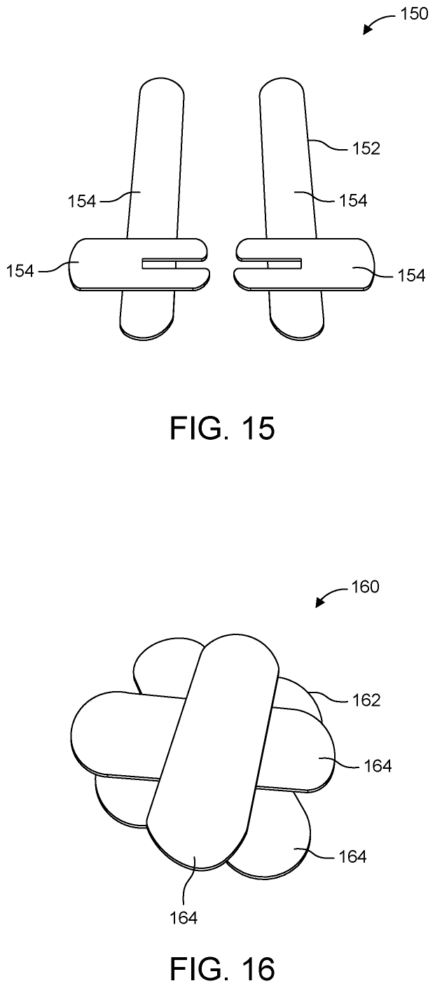

FIG. 15 is a plan view of one configuration 150 of the present disclosure that may be used to treat shin splints. Similar to configurations described above, one or more strip(s) 154 or shapes of pre-cut kinesiology tape may be provided (which may or may not be color-coded), or a single pre-cut shape of kinesiology tape may be provided. Several different types of essential oils may be useful for the encapsulated oil for the fabric layer 152. For example, the micro-encapsulated essential oils for this pre-cut kinesiology tape may be selected singly, or as a mixture from the group consisting of: rosehip, lavender, evening primrose, flaxseed, clary sage, eucalyptus, rosemary, juniper berry, chamomile, cayenne, sandalwood, clove, marjoram, yarrow, lemongrass, wintergreen, and peppermint.

FIG. 16 is a plan view of one configuration 162 of the present disclosure that may be used to treat muscle pain, such as muscle pain experienced when a spot on or near a strained muscle becomes a trigger point. Similar to configurations described above, one or more strip(s) 164 or shapes of pre-cut kinesiology tape may be provided (which may or may not be color-coded), or a single pre-cut shape of kinesiology tape may be provided. Several different types of essential oils may be useful for the encapsulated oil for the fabric layer 162. For example, the micro-encapsulated essential oils for this pre-cut kinesiology tape may be selected singly, or as a mixture from the group consisting of: rosehip, lavender, evening primrose, flaxseed, clary sage, eucalyptus, rosemary, juniper berry, chamomile, cayenne, sandalwood, clove, marjoram, yarrow, lemongrass, wintergreen, and peppermint.

In use, a user may first determine the purpose for the kinesiology tape, including the specific body area and treatment. For example, pain relief, recovery, muscle inhibition, tension reduction/relaxation, etc. The user may then select the shape of pre-cut kinesiology tape to achieve their purpose for that part of the body. For shipping and storage, the kinesiology tape may be provided with a protective layer over the adhesive layer to preclude the adhesive from being used before desired, such as a waxed or non-stick paper. When the user is ready to apply the tape, they may remove this protective layer from the adhesive layer, and apply the tape to the skin. If further desired, the user may continue with activities such as daily activities, work-outs, etc. Depending on the type of microencapsulation, such activities may assist in dispersing the essential oil(s) embedded within the fabric layer of the kinesiology tape as the tape is moved, or as friction acts against the tape, rupturing the microcapsules and causing further distribution of the embedded essential oils. Further release or distribution of the embedded essential oils may be triggered by friction against the kinesiology tape, such as by rubbing the tape.

According to one aspect, a kinesiology tape is disclosed, comprising: a fabric layer, the fabric layer comprising a weave of fibers, the fibers comprising at least some elastic fibers covered in cotton, and wherein the fabric layer further comprises at least one microencapsulated essential oil having activity such as anti-inflammatory, tension-reducing, and pain-reducing; an adhesive layer connected to the fabric layer, the adhesive layer in some instances comprising one or more strips or other configurations of adhesive connected to the fabric layer such that the adhesive layer does not in some instances cover the fabric layer entirely; wherein the kinesiology tape may be provided as a strip, a pre-cut strip, on a roll, or as a pre-cut shape, the pre-cut shape comprising one of an I-shape, a Y-shape, a V-shape, an L-shape, and an X-shape.

The pre-cut kinesiology tape may be provided in a kit containing one or more strips or shapes of kinesiology tape, such as a kit to treat upper back pain, lower back pain, shin splints, congestion, headaches, etc.

The adhesive layer may include a first strip of adhesive attached proximal to a first side of the kinesiology tape and a second strip of adhesive attached proximal to a second side. The microencapsulated essential oil may be embedded in the fabric layer.

In some configurations, the microencapsulated essential oil is selected singly, or as a mixture from the group consisting of: rosehip, lavender, evening primrose, flaxseed, clary sage, eucalyptus, rosemary, juniper berry, chamomile, cayenne, sandalwood, clove, marjoram, yarrow, lemongrass, wintergreen, and peppermint.

In other configurations, a kinesiology tape is described which may comprise: a flexible fabric layer, and wherein the flexible fabric layer further comprises at least two microencapsulated essential oils having anti-inflammatory activity; an adhesive layer connected to the fabric layer, the adhesive layer comprising one or more strips of adhesive connected to the fabric layer such that the adhesive layer does not cover the fabric layer entirely; wherein the kinesiology tape has a pre-cut shape, the pre-cut shape comprising an oval shape with one or more cut-outs. The adhesive layer may include a first strip of adhesive attached proximal to a first edge of the kinesiology tape and a second strip of adhesive attached proximal to a second edge. In some configurations, at least two microencapsulated essential oils are embedded in the flexible fabric layer. The microencapsulated essential oils may be selected singly, or as a mixture from the group consisting of: thyme, clove, rose, fennel, bergamot, peppermint, eucalyptus, lavender, chamomile, spruce, ginger, patchouli, and helichrysum.

According to another aspect, a method of treating muscle pain is provided, the method comprising: providing a kit of one or more pre-cut shapes of kinesiology tape, the one or more pre-cut shapes of kinesiology tape being selected from: an I-shape, a Y-shape, a V-shape, an L-shape, and an X-shape, and wherein the kinesiology tape comprises a flexible woven fabric layer and an adhesive layer; and embedding the fabric layer of the pre-cut portion of kinesiology tape singly, or as a mixture with at least one of the following micro-encapsulated essential oils: peppermint, helichrysum, ginger, black pepper, clove, eucalyptus, rosemary, and marjoram.

The method may also include the step of applying the pre-cut portion of kinesiology tape proximal to a muscle having pain. The method may also include the step of increasing body movement to release one or more of the microencapsulated essential oils. The method may also include the step of rubbing or applying friction to the kinesiology tape to release one or more of the microencapsulated essential oils. This step may be repeated throughout the wearing of the applied kinesiology tape to cause the kinesiology tape to release one or more of the microencapsulated essential oils.

According to another aspect, a method of manufacturing kinesiology tape is disclosed, the method comprising: selecting and providing a fabric layer, the fabric layer comprising a material such as a weave of fibers, the fibers comprising at least some elastic fibers covered in a covering; attaching an adhesive layer to a skin-facing side of the fabric layer, the adhesive layer in some instances comprising one or more strips of adhesive connected to the fabric layer such that the adhesive layer has a first width and the fabric layer has a second width, the second width being greater than the first width; cutting the fabric layer and adhesive layer into a pre-cut shape selected for its ability to reduce at least one of the following: pain, inflammation, and tension; embedding the fabric layer with at least one microencapsulated essential oil either as a single oil or as a mixture, the oil having an activity such as anti-inflammatory, tension-reducing, and pain-reducing. In some configurations, the method includes the step of cutting the fabric layer and adhesive layer into a pre-cut shape comprises cutting the fabric layer and adhesive layer into one of the following shapes: an elongate strip, a premeasured strip of any of a range of sizes, a perforated strip, an I-shape, a Y-shape, a V-shape, an L-shape, and an X-shape. In other configurations, a kit of one or more rolls and/or strips of tape is provided. In some instances, a covering for the elastic fibers may comprise cotton.

In some configurations, the pre-cut shape comprises an X-shape or a star-shape such as 60 of FIG. 4 or 160 of FIG. 16, and the step of embedding the fabric layer with at least one microencapsulated essential oil comprises embedding the fabric layer with at least one of the following essential oils singly or as a mixture: rosehip, lavender, evening primrose, flaxseed, clary sage, eucalyptus, rosemary, juniper berry, chamomile, cayenne, sandalwood, clove, marjoram, yarrow, lemongrass, wintergreen, and peppermint.

The various embodiments described above, including elements of the various embodiments described above, can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled.

1. A kinesiology tape comprising:
a flexible fabric layer, the fabric layer comprising a weave of fibers, the fibers comprising at least some elastic fibers covered in cotton, and wherein the fabric layer further comprises at least one microencapsulated essential oil;
an adhesive layer connected to the fabric layer, the adhesive layer comprising one or more strips of adhesive connected to the fabric layer such that the adhesive layer does not cover the fabric layer entirely;
wherein the kinesiology tape has a pre-cut shape, the pre-cut shape comprising one of an elongate strip for cutting, a pre-cut strip, a pre-sized strip, a perforated strip, a spooled strip, a premeasured strip, an I-shape, a Y-shape, a V-shape, an L-shape, a star shape, and an X-shape.

2. The kinesiology tape of claim 1, wherein the adhesive layer comprises a first strip of adhesive attached proximal to a first side of the kinesiology tape and a second strip of adhesive attached proximal to a second side.

3. The kinesiology tape of claim 1, wherein the microencapsulated essential oil is embedded in the fabric layer.

4. The kinesiology tape of claim 1, wherein the microencapsulated essential oil is selected from the group consisting of: rosehip, lavender, evening primrose, wintergreen, helichrysum, menthol, blue chamomile, flaxseed, Osmanthus, clary sage, ylang ylang, eucalyptus, rosemary, blue tansy, camphor, juniper berry, copaiba (such as copaifera reticulata, copaifera officinalis, copaifera langsdorffii and/or copaifera coriacea), chamomile, cayenne, sandalwood, clove, marjoram, yarrow, lemongrass, wintergreen, and peppermint.

5. A kinesiology tape comprising:
a flexible fabric layer, and wherein the flexible fabric layer further comprises at least two microencapsulated essential oils;
an adhesive layer connected to the fabric layer, the adhesive layer comprising one or more strips of adhesive connected to the fabric layer such that the adhesive layer does not cover the fabric layer entirely;
wherein the kinesiology tape has a pre-cut shape, the pre-cut shape comprising an oval shape with one or more cut-outs.

6. The kinesiology tape of claim 5, wherein the adhesive layer comprises a first strip of adhesive attached proximal to a first edge of the kinesiology tape and a second strip of adhesive attached proximal to a second edge.

7. The kinesiology tape of claim 5, wherein the at least two microencapsulated essential oils are embedded in the flexible fabric layer.

8. The kinesiology tape of claim 5, wherein the at least two microencapsulated essential oils are selected from the group consisting of: thyme, clove, rose, fennel, bergamot, peppermint, eucalyptus, lavender, chamomile, spruce, ginger, patchouli, and helichrysum.

9. A method of treating muscle pain, the method comprising:
providing one or more pre-cut shapes of kinesiology tape, the one or more pre-cut shapes of kinesiology tape being selected from: an elongate strip for cutting, a pre-cut strip, a pre-sized strip, a perforated strip, a spooled strip, a premeasured strip, an I-shape, a Y-shape, a V-shape, an L-shape, a star shape, and an X-shape, and wherein the kinesiology tape comprises a flexible woven fabric layer and an adhesive layer; wherein the kinesiology tape is embedded with at least one of the following microencapsulated essential oils: peppermint, helichrysum, ginger, black pepper, clove, eucalyptus, rosemary, and marjoram; and
applying at least one of the pre-cut shapes of kinesiology tape to a subject in need thereof.

10. The method of claim 9, wherein the step of applying at least one of the pre-cut shapes of kinesiology tape includes applying the kinesiology tape proximate to a muscle having pain.

11. The method of claim 10, further comprising the step of increasing body movement to release one or more of the micro-encapsulated essential oils.

12. The method of claim 9, further comprising the step of periodically rubbing the kinesiology tape to release the embedded microencapsulated essential oils.

13. A method of manufacturing kinesiology tape, the method comprising:
selecting a fabric layer, the fabric layer comprising a weave of fibers, the fibers comprising at least some elastic fibers covered in a covering;
attaching an adhesive layer to a distal side of the fabric layer, the adhesive layer comprising one or more strips of adhesive connected to the fabric layer such that the adhesive layer has a first width and the fabric layer has a second width, the second width being greater than the first width;
cutting the fabric layer and adhesive layer into a pre-cut shape selected for its ability to reduce at least one of the following: pain, inflammation, and tension;
embedding the fabric layer with at least one microencapsulated essential oil.

14. The method of claim 13, wherein the step of cutting the fabric layer and adhesive layer into the pre-cut shape comprises cutting the fabric layer and adhesive layer into one of the following shapes: an I-shape, a Y-shape, a V-shape, an L-shape, a star shape, and an X-shape.

15. The method of claim 13, wherein the covering for the elastic fibers comprises cotton.

16. The method of claim 14, wherein the pre-cut shape comprises an X-shape and wherein the step of embedding the fabric layer with at least one microencapsulated essential oil comprises embedding the fabric layer with at least one of the following essential oils: rosehip, lavender, evening primrose, flaxseed, clary sage, eucalyptus, rosemary, juniper berry, chamomile, cayenne, sandalwood, clove, marjoram, yarrow, lemongrass, wintergreen, and peppermint.

17. The method of claim 14, further comprising the step of providing two or more strips of pre-cut kinesiology tape in a kit.
